(19) United States
(12) Patent Application Publication
(10) Pub. No.: US 2023/0245714 A1
(43) Pub. Date: Aug. 3, 2023

(54) DYNAMICALLY RESIZED SLIDING WINDOW FOR VARIANT ANALYSIS

(71) Applicant: Helix OpCo, LLC, San Mateo, CA (US)

(72) Inventors: Elizabeth Cirulli Rogers, Lakeside, CA (US); Kelly Schiabor Barrett, San Diego, CA (US); Nicole Washington, Albany, CA (US)

(21) Appl. No.: 17/575,894

(22) Filed: Jan. 14, 2022

(51) Int. Cl. G16B 20/20; G16B 30/00

(52) U.S. Cl. CPC G16B 20/20; G16B 30/00

(57) ABSTRACT

Systems and methods are provided for analysis of genetic data. One embodiment is a system that includes a memory storing sequence data and trait data. The system also includes a controller that identifies qualifying variants within the sequence data. The controller generates a sliding window comprising a selection of sequential variants, wherein a number of the individuals in the population carrying a qualifying variant at the sliding window is within a predetermined range, and iteratively: performs a statistical analysis that indicates whether qualifying variants at genomic coordinates within a region occupied by the sliding window are correlated with the trait, and moves the sliding window across at least one variant along a chromosomal direction, while adjusting a number of variants encompassed by the sliding window to maintain a number of the individuals in the population carrying a qualifying variant at the sliding window within the predetermined range.

### FIELD

The disclosure relates to the field of genomic analysis, and in particular, to identifying and reporting the variants of a population of individuals that are associated with a specific trait.

### BACKGROUND

A common technique utilized to identify associations between genetic variants and traits (e.g., phenotypes) of individuals is known as a Genome-Wide Association Study (GWAS). GWAS is performed by acquiring genomic data and trait data for a population, and then identifying genetic variants within the population that are statistically associated with the trait being considered. Genetic variants that appear with a greater (or lesser) allele frequency within the portion of the population expressing the trait than across the entire population are expected to be associated positively (or negatively) with the trait.

GWAS is subject to numerous limitations in terms of precision. To address these limitations, particularly with regard to variants that are rare, gene-based collapsing analysis is utilized instead of GWAS. Gene-based collapsing analysis considers a small portion of the genome at a time for statistical analysis, rather than analyzing each genetic variant individually. Even so, selecting the exact portion of the genome to be considered at once for statistical purposes remains a difficult task. This is particularly notable when attempting to determine the influence of a rare variant on a trait, as common variants can mask the influence of nearby rare variants.

Hence, scientists and medical practitioners continue to seek out enhanced systems and methods for detecting genetic variations associated with traits in a precise, accurate, and computationally efficient manner.

### SUMMARY

Embodiments described herein implement a sliding window approach, wherein the size (along a portion of a chromosome) of a window of variants considered for statistical analysis dynamically varies. Specifically, the size of the window (e.g., in variants, bases, exons, etc.) is scaled to maintain a consistent range of individuals carrying qualifying variants as the window advances through genomic coordinates. Hence the number of variants encompassed by the window is dynamically adjusted as analysis is performed. The systems and methods described herein provide a technical benefit over prior techniques, because they enable greater statistical consistency when attempting to determine the influence of rare variants with regard to specific conditions.

One embodiment is a system that includes a memory that stores sequence data for a portion of a chromosome that indicates, for each individual in a population, variants found in the individual within the portion of the chromosome, and trait data indicating, for each of the individuals, an extent that the individual expresses a predefined trait controlled by the portion of the chromosome. The system also includes a controller able to identify qualifying variants within the sequence data. The qualifying variants include variants that meet criteria for analysis. For each qualifying variant, the controller is able to determine a genomic coordinate of the qualifying variant at the portion of the chromosome, as well as a number of the individuals in the population carrying the qualifying variant. The controller is further able to generate a sliding window comprising a selection of a sequential set of variants within the portion of the chromosome, wherein a number of the individuals in the population carrying a qualifying variant at the sliding window is within a predetermined range, and to iteratively: perform a statistical analysis that indicates whether qualifying variants at genomic coordinates within a region occupied by the sliding window are correlated with the trait, based on a comparison of trait data for individuals carrying the qualifying variant to trait data for individuals in the population, and move the sliding window across at least one variant along a chromosomal direction, while dynamically adjusting a number of variants encompassed by the sliding window to maintain a number of the individuals in the population carrying a qualifying variant at the sliding window within the predetermined range. The controller is further able to selectively categorize qualifying variants as correlated with the trait, based on the statistical analysis, and to report qualifying variants correlated with the trait to a user via a display.

A further embodiment is a method. The method includes storing sequence data for a portion of a chromosome that indicates, for each individual in a population, variants found in the individual within the portion of the chromosome, and storing trait data indicating, for each of the individuals, an extent that the individual expresses a predefined trait controlled by the portion of the chromosome. The method also includes identifying qualifying variants within the sequence data, the qualifying variants comprising variants that meet criteria for analysis, and for each qualifying variant, determining a genomic coordinate of the qualifying variant at the portion of the chromosome, as well as a number of the individuals in the population carrying the qualifying variant. The method also includes generating a sliding window comprising a selection of a sequential set of variants within the portion of the chromosome, wherein a number of the individuals in the population carrying a qualifying variant at the sliding window is within a predetermined range. The method further includes iteratively: performing a statistical analysis that indicates whether qualifying variants at genomic coordinates within a region occupied by the sliding window are correlated with the trait, based on a comparison of trait data for individuals carrying the qualifying variant to trait data for individuals in the population, and moving the sliding window across at least one variant along a chromosomal direction, while dynamically adjusting a number of variants encompassed by the sliding window to maintain a number of the individuals in the population carrying a qualifying variant at the sliding window within the predetermined range. The method further includes selectively categorizing qualifying variants as correlated with the trait, based on the statistical analysis; and reporting qualifying variants correlated with the trait to a user via a display.

A further embodiment is a non-transitory computer readable medium embodying programmed instructions which, when executed by a processor, are operable for performing a method. The method includes storing sequence data for a portion of a chromosome that indicates, for each individual in a population, variants found in the individual within the portion of the chromosome, and storing trait data indicating, for each of the individuals, an extent that the individual expresses a predefined trait controlled by the portion of the chromosome. The method also includes identifying qualifying variants within the sequence data, the qualifying variants comprising variants that meet criteria for analysis, and for each qualifying variant, determining a genomic coordinate of the qualifying variant at the portion of the chromosome, as well as a number of the individuals in the population carrying the qualifying variant. The method also includes generating a sliding window comprising a selection of a sequential set of variants within the portion of the chromosome, wherein a number of the individuals in the population carrying a qualifying variant at the sliding window is within a predetermined range. The method further includes iteratively: performing a statistical analysis that indicates whether qualifying variants at genomic coordinates within a region occupied by the sliding window are correlated with the trait, based on a comparison of trait data for individuals carrying the qualifying variant to trait data for individuals in the population, and moving the sliding window across at least one variant along a chromosomal direction, while dynamically adjusting a number of variants encompassed by the sliding window to maintain a number of the individuals in the population carrying a qualifying variant at the sliding window within the predetermined range. The method further includes selectively categorizing qualifying variants as correlated with the trait, based on the statistical analysis; and reporting qualifying variants correlated with the trait to a user via a display.

Other illustrative embodiments (e.g., methods and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a block diagram depicting a variant analysis system that inspects sequence data and trait data in an illustrative embodiment.

FIG. 2 is a flowchart depicting a method of operating a variant analysis system in an illustrative embodiment.

FIG. 3 depicts a table that summarizes sequence data for individuals in an illustrative embodiment.

FIG. 4 depicts a table that summarizes trait data for individuals in an illustrative embodiment.

FIG. 5 depicts variants arranged along a portion of a chromosome with exons in an illustrative embodiment.

FIG. 6 depicts a sliding window that slides across the portion of the chromosome in an illustrative embodiment.

FIG. 7 depicts positions of the sliding window that are positively associated with a trait being considered in an illustrative embodiment.

FIG. 8 depicts a mapping of positions of the sliding window to variants in an illustrative embodiment.

FIG. 9 depicts a Graphical User Interface (GUI) that provides a report for review by a researcher in an illustrative embodiment.

FIG. 10 depicts an illustrative computing system operable to execute programmed instructions embodied on a computer readable medium.

### DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

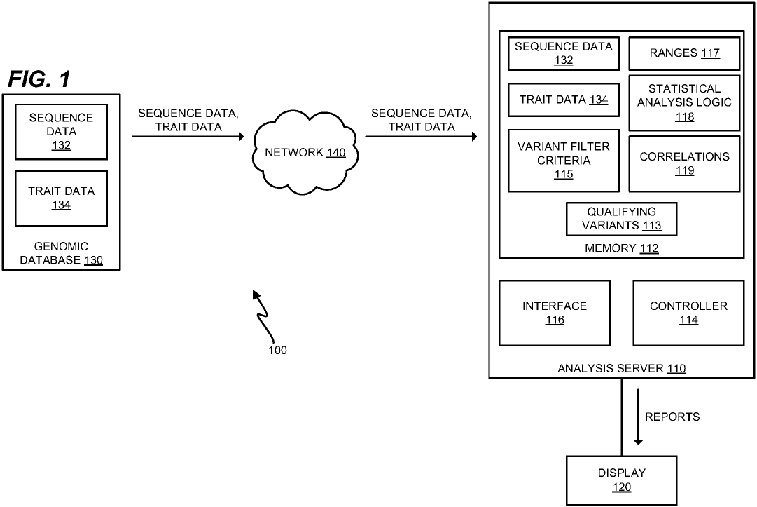

FIG. 1 is a block diagram depicting a variant analysis system 100 that inspects sequence data and trait data in an illustrative embodiment. Variant analysis system 100 comprises any system, components, and/or devices that are capable of determining the influence of variants (e.g., variations in genetic information carried by individual persons) on traits, based on sequence data and trait data. Specifically, variant analysis system 100 may identify rare variants that are statistically correlated with a known trait. As used herein, a variant comprises a unique combination of genetic information, in the form of consecutive base pairs at a specific set of locations (e.g., genomic coordinates) along a portion of a chromosome. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, insertions and/or deletions (Indels) which relate for example to the insertion or deletion of less than thirty base pairs, or differing numbers of repetitions, copy number variants, which relate to larger insertions or deletions, translocations, inversions, other types of genetic variants, or even combinations of variants, such as haplotypes or Multi-nucleotide variants (MNVs). A rare variant comprises an alternative form of genetic information that is present with a low Minor Allele Frequency (MAF), such as a MAF of less than one percent.

Variant analysis system 100 generates and moves a sliding window across a portion of a chromosome to detect rare variants correlated with the trait being investigated. The sliding window groups variants located near each other into one unit, and analyzes them together to improve statistical power, much like a gene-based collapsing analysis, but at a smaller scale.

In this embodiment, variant analysis system 100 includes an analysis server 110, which communicates with a genomic database 130 via a network 140 (e.g., the Internet, a private network, a wireless or wired network, etc.) to retrieve sequence data 132 and trait data 134. Sequence data 132 comprises any suitable information for reporting the sequence of base pairs along a portion of a chromosome, for each of multiple individuals in a population (e.g., for each of hundreds of thousands of individuals). For example, sequence data 132 may comprise files in common formats for genetic information, Variant Call Format (VCF) files, plink-formatted Browser Extensible Database (BED) files, etc.

Trait data 134 comprises any suitable information reporting the existence (or nonexistence) of specific traits (e.g., genetic diseases, phenotypes, etc.) within each of multiple individuals in the population. As used herein, a trait may comprise a state of having a disease or condition, a phenotype, a diagnosis indicated in a health record, an International Classification of Diseases (ICD) code indicated in a health record, a quantitative trait measurement such as but not limited to height or total cholesterol levels in blood, or some combination thereof. In one embodiment, trait data 134 is derived by analysis server 110 from a collection of Electronic Health Records (EHRs) or other medical records.

Although sequence data 132 and trait data 134 are depicted as being distinct pieces of information stored at genomic database 130, in further embodiments the sequence data 132 and trait data 134 may be intermixed with each other, or may be sourced from different entities. Furthermore, sequence data 132 and/or trait data 134 may include common identifiers for the same individuals in the population, in order to facilitate associations between sequence data 132 and trait data 134.

After interface 116 (e.g., a wired or wireless ethernet interface) of analysis server 110 has retrieved the sequence data 132 and the trait data 134, controller 114 stores both in memory 112. Controller 114 may additionally process the sequence data 132 and the trait data 134 to prepare them for analysis. For example, controller 114 may review the variant filter criteria 115 to identify qualifying variants within the sequence data 132. As used herein, a qualifying variant is a variant selected for study by controller 114, in order to determine whether a correlation exists between that variant and the trait being considered. Thus, a qualifying variant is often, but not always, a rare variant.

In one embodiment, variant filter criteria 115 indicates that any variant which alters a structure of a protein (e.g., a protein generated by the portion of the chromosome) is a qualifying variant. Controller 114 may then store the qualifying variants 113 in memory 112, along with identifying information for those qualifying variants. Such information may comprise a number of persons in the population carrying the qualifying variant, a list reciting individuals carrying the qualifying variant, a location of each qualifying variant within a portion of a chromosome, a sequence of each qualifying variant, etc. Controller 114 may be implemented, for example, as custom circuitry, as a hardware processor executing programmed instructions, or some combination thereof.

Memory 112 also stores ranges 117, statistical analysis logic 118, and correlations 119. Ranges 117 indicate a number of individuals to include within a sliding window. In one embodiment, ranges 117 comprise a numerical range, such as from twenty to sixty, or from ten to one thousand. In further embodiments, ranges 117 each comprise a single number, and the sliding window is permitted to adjust its size to any size within a predefined distance of the single number. In still further embodiments, the range is determined to provide a predetermined power level. For example, the range may include a number which ensures that the odds ratio and/or p-value are calculated to a predetermined margin of error (e.g., providing eighty percent power to identify an odds ratio of at least 4) for each position of the sliding window.

Statistical analysis logic 118 comprises instructions in memory 112 which are utilized by controller 114 to determine whether or not a correlation exists between the variants in a sliding window and the trait being investigated. In one embodiment, statistical analysis logic 118 includes code for one or more equations that quantify the amount of correlation detected for the sliding window, at each position along a portion of a chromosome occupied by the sliding window. Such equations may determine an odds ratio, a p-value, and/or other statistical measures.

Correlations 119 comprise information maintained by controller 114 indicating the existence and/or strength of correlation between specific variants (or groups of variants) and specific traits. For example, correlations 119 may indicate, for each qualifying variant that has been studied, an odds ratio and/or a p-value for the qualifying variant in relation to the trait being considered.

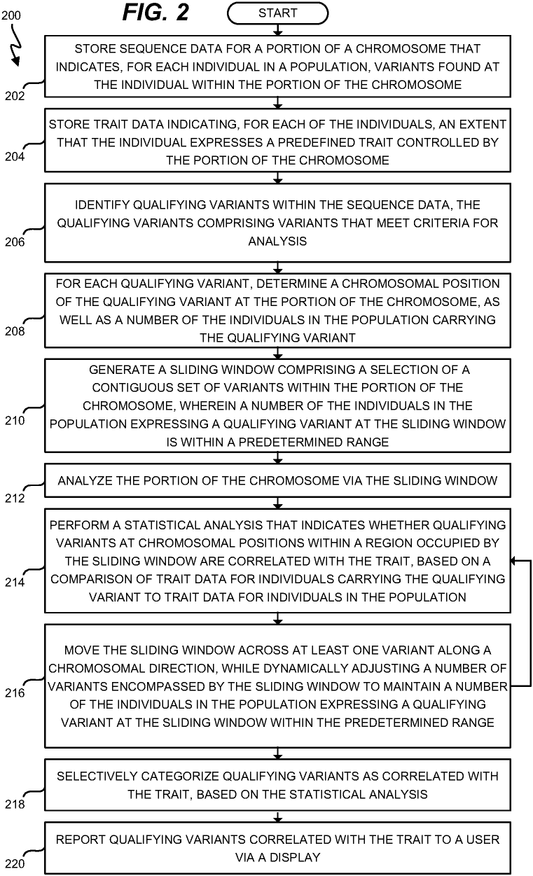

Illustrative details of the operation of variant analysis system 100 will be discussed with regard to FIG. 2. Assume, for this embodiment, that analysis server 110 is being initialized by an operator that wishes to determine the influence of rare variants within a specific portion of a chromosome (e.g., corresponding with a specific gene, or a specific range of genomic coordinates) upon a trait.

FIG. 2 is a flowchart depicting a method of operating a variant analysis system 100 in an illustrative embodiment. The steps of method 200 are described with reference to variant analysis system 100 of FIG. 1, but those skilled in the art will appreciate that method 200 may be performed in other systems. The steps of the flowcharts described herein are not all inclusive and may include other steps not shown. The steps described herein may also be performed in an alternative order.

Step 202 includes storing sequence data 132 for a portion of a chromosome that indicates, for each individual in a population, variants found in the individual within the portion of the chromosome. Step 202 may include controller 114 querying genomic database 130 for sequence data 132 for specific populations (e.g., populations having specific demographics, populations that have known data for the trait being considered, etc.). Interface 116 receives the sequence data 132, and controller 114 stores the sequence data 132 in memory 112. In further embodiments, to facilitate bandwidth efficiency, controller 114 only requests the specific portion of the chromosome being considered.

Step 204 includes storing trait data 134 indicating, for each of the individuals, an extent that the individual expresses a predefined trait controlled by the portion of the chromosome. This step may be performed in tandem with step 202 and integrated into the communications discussed therein, or may alternatively be performed as a separate step by controller 114. For binary traits that either exist or do not exist, an extent of expression may be an indication that the trait exists or does not exist. For traits that are quantitative in nature (e.g., height), an extent may indicate the degree or amount of expression of the trait.

In further embodiments, access to genomic database 130, and/or specific compilations of sequence data 132 and/or trait data 134, is restricted to specific credentials which may be provided by controller 114 based on user input, in order to provide enhanced security. In such embodiments, data sent over network 140 may be encrypted and/or anonymized to ensure privacy.

After controller 114 has stored both sequence data 132 and trait data 134 in memory 112, controller 114 may process the sequence data 132 and the trait data 134 to associate specific trait information with specific variant information for individuals in the population. For example, controller 114 may generate a set of associations between sequence data 132 and trait data 134 for each individual, based on common identifiers found therein.

Step 206 includes controller 114 identifying qualifying variants within the sequence data 132. The qualifying variants comprise variants that meet criteria for analysis and are also within the portion of the chromosome selected for analysis. Identifying qualifying variants may comprise reviewing the sequence data 132 of each individual, based on variant filter criteria 115 in memory 112. For example, variant filter criteria 115 may define qualifying variants as variants that are coding (e.g., having base pairs that indicate stop_lost, mis-sense_variant, start_lost, splice_donor_variant, inframe_deletion, frameshift_variant, splice_acceptor_variant, stop_gained, or inframe_insertion) and also are not Polyphen benign or Sorting Intolerant From Tolerant (SIFT) benign. In such an embodiment, Polyphen benign may be considered any value less than 0.15, while SIFT benign may be considered any value that is greater than 0.05. In a further example, similar sequence pathogenicity algorithms may be utilized that are field-standard and used for such a purpose.

In a still further embodiment, variant filter criteria 115 may define qualifying variants as Loss of Function (LoF) variants (e.g., having base pairs that indicate stop_lost, start_lost, splice_donor_variant, frameshift_variant, splice_acceptor_variant, or stop_gained) or variants having other predicted molecular properties, such as mis-sense (a change in corresponding amino acid), splice site variants, etc. In such an embodiment, a variant may be required to be below a MAF cutoff of 0.1% in all Genome Aggregation Database (gnomAD) populations, locally within each population analyzed (e.g., in populations representative of African, East Asian, European, South Asian, and Hispanic descent), in order to be considered a qualifying variant.

Although the discussion herein focuses upon qualifying variants as variants that impact the protein sequence of a gene, this is not a requirement of the method, and the same technique may be applied in introns or intergenic regions, sliding along genomic coordinates that are outside of genes.

Step 208 comprises controller 114 determining genomic coordinate(s) for each qualifying variant at the portion of the chromosome being considered. This operation may be performed by controller 114 during step 206, each time a new qualifying variant is detected. For example, each time a qualifying variant is detected by controller 114, controller 114 may record the genomic coordinate(s) occupied by the qualifying variant.

Controller 114 also determines a number of the individuals in the population carrying the qualifying variant. This may be performed by controller 114 counting individuals carrying qualifying variants that occupy the same genomic coordinates within the population, counting individuals carrying qualifying variants that have identical sequences at the same genomic coordinates, etc. Controller 114 may even generate a table that reports the sequences of qualifying variants within the population, the genomic coordinate(s) of each qualifying variant, the number of individuals carrying each qualifying variant, and the number of individuals carrying each qualifying variant that also express the trait. This information helps to fuel the statistical analysis process described below.

Step 210 includes generating a sliding window comprising a selection of a sequential (e.g., adjacent, contiguous, etc.) set of variants within the portion of the chromosome. In further embodiments, the sliding window may comprise a range of sequential base pairs, exons, variants, etc., within the portion of the chromosome, or a bounded range of genomic coordinates. The number of the individuals in the population carrying a qualifying variant at the sliding window will be maintained by controller 114 within a predetermined range as the sliding window advances across the portion of the chromosome. This ensures that the strength of statistical correlations made between variants and the trait within the sliding window remain broadly similar, which facilitates the process of comparing the results of statistical analysis across the portion of the chromosome. In many embodiments, the base pairs, exons, variants, etc. encompassed by the sliding window at a given position are all contiguous with each other, with the exception that in some cases, variants etc. having more than a threshold number of carriers are pulled out for independent statistical analysis.

The size of the sliding window is defined based on the number of individuals carrying a qualifying variant that are within the range of positions occupied by the sliding window on the portion of the chromosome. That is, the sliding window extends from an initial position on the portion of the chromosome to a position where it encompasses a number of individuals carrying qualifying variants within the predetermined range. At each position, the sliding window encompasses a region that may be defined by a front border (with respect to the direction of travel of the sliding window) and a rear border.

Step 212 includes analyzing the portion of the chromosome via the sliding window to detect correlations between qualifying variants and the trait. This comprises iteratively performing steps 214-216, described below.

Step 214 includes controller 114 performing a statistical analysis that indicates whether qualifying variants at genomic coordinates within a region occupied by the sliding window are correlated with the trait, based on a comparison of trait data for individuals carrying the qualifying variant to trait data for individuals in the population.

In one embodiment, step 214 is performed by controller 114 determining a first ratio of individuals expressing the trait among individuals carrying a qualifying variant within the sliding window, determining a second ratio of individuals expressing the trait among the population, and determining an odds ratio based on: a comparison of the first ratio to the second ratio, a number of individuals carrying a qualifying variant within the sliding window, a number of individuals in the population, and consideration of covariates that affect the predefined trait. In one embodiment, covariates considered by controller 114 include age, sex, age*sex, age*age, sex*age*age, and the bioinformatics pipeline version being used by genomic database 130. A p-value may also be calculated using the same data that was used to calculate odds ratio or Beta value, with the assumption of a random distribution of the trait in the population.

In one embodiment, if the odds ratio is greater than 2.9 and the p-value is less than 0.05, then qualifying variants within the sliding window are considered correlated with the trait. In a further embodiment, if the odds ratio is greater than 4 and the p-value is less than 0.001, then the qualifying variants within the sliding window are considered correlated with the trait.

In one embodiment, controller 114 analyzes the sliding window for the presence of high Percent Spliced In (hiPSI) exons. PSI is a measure of splicing derived from the RNA-seq sequencing technique, and is an estimate of the percentage of transcripts that incorporate a given exon. A PSI of one hundred percent implies a consistently expressed exon. In certain cases, hiPSI exons may confound statistical analyses. To that end, sequence data for non-hiPSI exons may be ignored by controller 114 or otherwise excluded from the statistical analysis process. Pathogenicity models may also be updated to eliminate hiPSI exons that are not correlated with the trait, such as by excluding positions of the sliding window where the p-value is not less than 0.001. Effectively, the consideration of hiPSI exons acts as another filter for qualifying variants. This layers on information from other genomic-based molecular assays (e.g., RNA-seq) to further limit which variants qualify for analysis by controller 114. For example, hiPSI data may be relevant because it represents that subset of exons of a gene that are actually expressed (i.e., in RNA) in desired portions of tissue related to the trait being considered (e.g., cardiac tissue). In another embodiment, variants are only considered qualifying by controller 114 if they lie within sections of the gene or genome that are annotated as belonging to a certain functional or structural domain, such as a promoter region or a transmembrane domain.

In further embodiments, statistical tools such as the regenie code library for regression modelling facilitate the analysis process. In short, regenie is regression software that facilitates the quantitative determination of odds ratios and p-values. Such regression software also helps to control for covariates. Controller 114 may utilize regenie or other tools to build a whole genome regression model or other statistical model, using common variants (e.g., variants that do not meet the definition of rare variants) to account for the effects of relatedness and population stratification. Controller 114 may further use regenie or other analysis software tools to perform regression or other statistical analysis (e.g., Fisher’s exact test or a Cox proportional hazards model) for individual positions of the sliding window. Such tools may also be utilized to account for situations where case-control imbalance exists. Case-control imbalance leads to test statistic inflation with standard logistic regression analysis methods.

Regression or other statistical analysis may also be performed by controller 114. For example, for binary variables, logistic regression may be performed, while for count data, negative binomial regression may be used. In further embodiments, for quantitative variables, linear regression is used by controller 114 after rank-based inverse normal transformation of the variable. Time-to-event analyses, and/or other analyses, may be performed by controller 114 using the lifelines KaplanMeier package in the python coding language.

The statistical analysis performed by controller 114 may also include bootstrapping. Bootstrapping is a process by which a subset of a total dataset is sampled for analysis, and the process is repeated by sampling multiple times with replacement. For example, for an individual position of a sliding window being analyzed according to method 200, with a total sample size of two hundred thousand, the analysis could be run ten times. The first time would involve controller 114 randomly sampling one hundred thousand individuals from the set of two hundred thousand, running the analysis on just the sample, and recording the results. The second time, controller 114 samples another random one hundred thousand individuals from the set of two hundred thousand. This process continues with replacement--meaning that all two hundred thousand individuals are available to be randomly sampled into the set of one hundred thousand each time--until ten analyses of one hundred thousand individuals randomly sampled from the two hundred thousand have been completed. Information about the ten analyses, such as how often a p-value fell below 0.001, or a median odds ratio from the analyses, can then be used by controller 114 to identify whether a position of a window is of interest. This process may use any sample size and any number of bootstrapping iterations for the analysis (for example, ten thousand analyses per window instead of just ten).

Step 216 includes moving the sliding window across at least one variant along a chromosomal direction (e.g., from 3′ to 5′, or from 5′ to 3′ along the chromosome). Controller 114 performs this operation while dynamically adjusting a number of variants encompassed by the sliding window to maintain the number of individuals in the population carrying a qualifying variant within the sliding window at a predetermined range. This operation may be performed by stepping one border (e.g., a rear border) of the sliding window by a base pair, exon, or variant, from a first base pair, exon, or variant, to an adjacent base pair, exon, or variant (i.e., along the chromosomal direction), while stepping another border (e.g., a front border) of the sliding window by a variable number of base pairs, exons, variants, etc., to ensure that the population of individuals carrying a qualifying variant within the sliding window remains within the predetermined range. In one embodiment, the predetermined range is forty individuals.

Implementing a sliding window, wherein the sliding window exhibits a similar power for statistical significance for correlations along all of its positions, enables direct comparisons between regions of a portion of a chromosome that were not previously possible. This vastly improves the process of identifying and investigating variants that may have a notable role in relation to specific traits, because it ensures that time is not wasted comparing results that have notably different statistical underpinnings.

In a further embodiment, if a qualifying variant has more than a threshold number of carriers, the qualifying variant is passed over by the sliding window (e.g., the sliding window moves at least one of its borders past the qualifying variant, or simply omits data for the qualifying variant from the analysis process). That qualifying variant then receives its own independent analysis via controller 114 for statistical correlation with the trait being considered. Because that qualifying variant has a large enough number of carriers, an independent analysis is assured to be performed with the desired amount of statistical power (e.g., at least the same power as for analyses relating to the sliding window).

Step 218 includes selectively categorizing qualifying variants as correlated with the trait, based on the statistical analysis. In one embodiment, this comprises determining a p-value indicating a likelihood that the qualifying variants within the sliding window are correlated with the trait, and identifying all qualifying variants in positions of the sliding window that have a p-value that is less than a threshold (e.g., one thousandth) as being correlated with the trait.

Step 220 includes reporting qualifying variants correlated with the trait to a user via a display. Step 220 may be performed by controller 114 operating display 120 to present correlations to a researcher for further inspection and quantification. In this embodiment, display 120 is directly coupled with the analysis server 110. However, in further embodiments, display 120 is updated based on instructions provided by analysis server 110 to a user device (e.g., a laptop or mobile phone) via the network 140.

Method 200 provides a substantial advantage over prior techniques because it ensures that the statistical power (i.e., precision and/or accuracy) of each correlation determined for the sliding window, at each position of the sliding window along the portion of the chromosome, remains either constant or within a predefined range. This results in a stable likelihood of correlation accuracy, which enhances consistency and eliminates the need to re-analyze and/or re-acquire population data. Furthermore, method 200 enables analysis to be performed for numerous portions of a chromosome, and/or for numerous traits, asynchronously and/or in parallel, in a manner that would be impossible to perform as a mental process or by hand.

Controller 114 may perform an additional course of action after correlated qualifying variants have been detected. In one embodiment, the controller 114 compares the results of the statistical analysis for the sliding window at different regions (e.g., different variants, exons, base pairs), and identifies at least one region within the portion of the chromosome that is more highly correlated with the trait than other regions within the portion of the chromosome (e.g., by having the highest odds ratio or lowest p-value). The controller 114 then reports this region as the most promising region for further investigation via a GUI provided at display 120.

In a further embodiment, controller 114 reports qualifying variants that were associated with the trait for each position of the sliding window that encompassed those qualifying variants. That is, if the sliding window was associated with the trait for all positions that encompassed a specific qualifying variant, that specific qualifying variant is notably likely to be positively correlated with the trait. Similarly, if the sliding window was not associated with the trait for all positions that encompassed a specific qualifying variant, that specific qualifying variant is notably likely to not be correlated with the trait. If the sliding window was not associated with the trait for some positions of the sliding window that encompassed a specific qualifying variant, then deeper analysis may be required to come to a conclusion. Based on these results, controller 114 actively identifies and reports the most relevant qualifying variants for further research.

In another embodiment, controller 114 actively engages in validation of its statistical analysis in method 200. To this end, controller 114 alters the predetermined range to a different number of individuals (e.g., twenty or sixty individuals instead of forty), generates a new sliding window, and iteratively performs statistical analysis and moves the new sliding window according to the altered predetermined range. The controller 114 then reports qualifying variants correlated with the trait for both the predetermined range and the altered predetermined range. This technique ensures that statistical analysis of the portion of the chromosome is rigorous. In still further embodiments, the controller 114 repeats the analysis by moving the sliding window in a different chromosomal direction, selecting a related trait for analysis, etc., before reporting qualifying variants that are correlated according to both analyses.

In one embodiment, controller 114 automatically selects a new portion of a chromosome for analysis, based on the detected correlations. For example, if more than a threshold number of correlations have been detected with qualifying variants in a first portion of a chromosome, controller 114 may perform method 200 on an adjacent portion of the same chromosome. Alternatively, if less than the threshold number of correlations have been detected with qualifying variants, controller 114 may perform method 200 on a distant portion of the same chromosome, or a portion of another chromosome entirely.

In another embodiment, upon completing a list of qualifying variants correlated with the trait and detecting more than a threshold number of correlations, controller 114 performs method 200 for a related trait, in a related category. For example, the category of cardiac health may include separate traits of atrial fibrillation and arrythmia. Controller 114 may perform analyses for each separate trait, as well as a trait comprising the combination of all of the separate traits.

In yet another embodiment, controller 114 actively ranks the qualifying variants based on the strength of the correlation (e.g., from highest to lowest odds ratio) and sorts the qualifying variants in that order in the report, to facilitate rapid detection of notable qualifying variants.

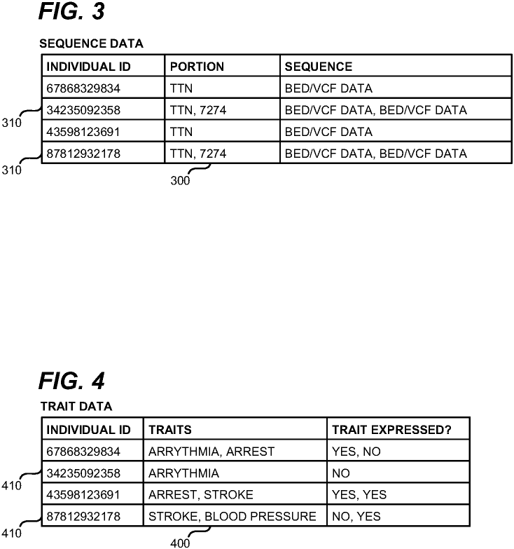

With a discussion provided above pertaining to determining associations between sequence data 132 and trait data 134, FIGS. 3-4 provide further context into the potential nature of these inputs to variant analysis system 100. FIG. 3 depicts a table 300 that summarizes sequence data 132 for individuals in an illustrative embodiment. In this embodiment, table 300 includes an entry 310 for each of multiple different individuals. Each entry 310 includes a unique identifier for the corresponding individual, as well as an indication of the portions of the genome for that individual that the sequence data 132 relates to. The portion of the genome may comprise whole genome data, whole exome data, array data, data for a specific gene or portion of a gene, etc. In this embodiment, the sequence data 132 for some individuals indicates multiple portions, for example including the TTN gene that codes for the protein titin, and the TTPA gene that is responsible for hereditary vitamin E deficiency. In further embodiments, sequence data 132 may be included for any suitable portions of the genome, or the entire genome.

Table 300 also includes a link to sequence data for each portion, as well as a format of the sequence data. As a general principle, the sequence data 132 indicates base pairs of the corresponding individual, together with the positions of those base pairs along a portion of a chromosome. In this embodiment, the sequence data 132 is stored as VCF and/or BED data, although any suitable technique for storing the sequence data 132 may be utilized. In this manner, a controller 114 of the analysis server 110 may use the table 300 to rapidly identify the variants carried by each individual in the population being studied. In further embodiments, controller 114 updates table 300 to list qualifying variants detected for each individual.

FIG. 4 depicts a table 400 that summarizes trait data 134 for individuals in an illustrative embodiment. In this embodiment, the table 400 includes multiple entries 410 for distinct individuals. Each entry 410 includes an identifier for the corresponding individual, a list of traits considered for expression by the individual, and data indicating whether the considered traits are expressed at the individual. In further embodiments, the data is not limited to mere binary indicators of expression, but also includes quantitative information (e.g., a number) indicating a prevalence or severity of the trait being considered.

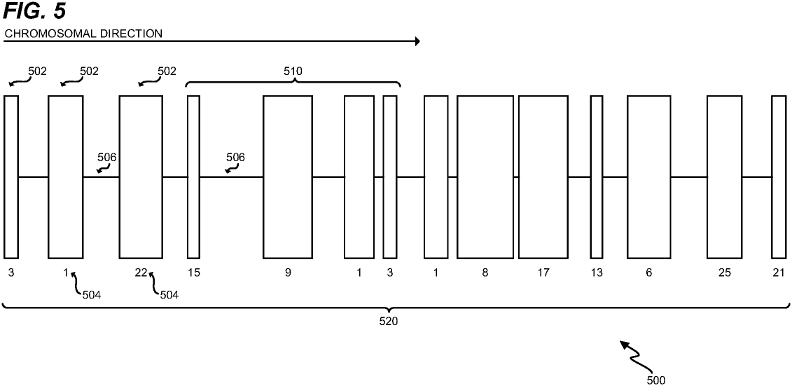

With FIGS. 3-4 pertaining to inputs for a variant analysis system 100 provided above, FIGS. 5-8 relate to the creation and travel of a sliding window across a portion of a chromosome in an illustrative embodiment. In this embodiment, FIGS. 5-8 depict a sliding window that advances across exons of a portion of a chromosome, but the same techniques may be utilized to move the window by other amounts. Thus, references to an “exon” with regard to FIGS. 5-7 may similarly be applied with respect to a variant, a base pair, etc. as a matter of design choice.

FIG. 5 depicts variants arranged along a portion 520 of a chromosome 500 in an illustrative embodiment. In this embodiment, the portion 520 includes multiple exons 502, which may be separated by introns 506. The portion 520 may also be subdivided into regions 510 of any suitable size (e.g., in base pairs, exons, variants, etc.). The exons 502 may each be associated with a certain number of variants.

For each exon 502 in the portion 520, memory 112 of analysis server 110 stores information indicating a number of individuals carrying a qualifying variant for that exon 502. This is represented via labels 504. The number of individuals need not carry the same qualifying variant for the exon 502. Rather, any qualifying variant at the exon 502 will suffice.

Labels 504 make clear that while each exon 502 may be carried by the entire population being considered (e.g., hundreds of thousands or millions of people), controller 114 is primarily focused on the number of individuals in the population that carry a qualifying variant for an exon 502. It is this number of individuals carrying qualifying variants that will be considered during analysis.

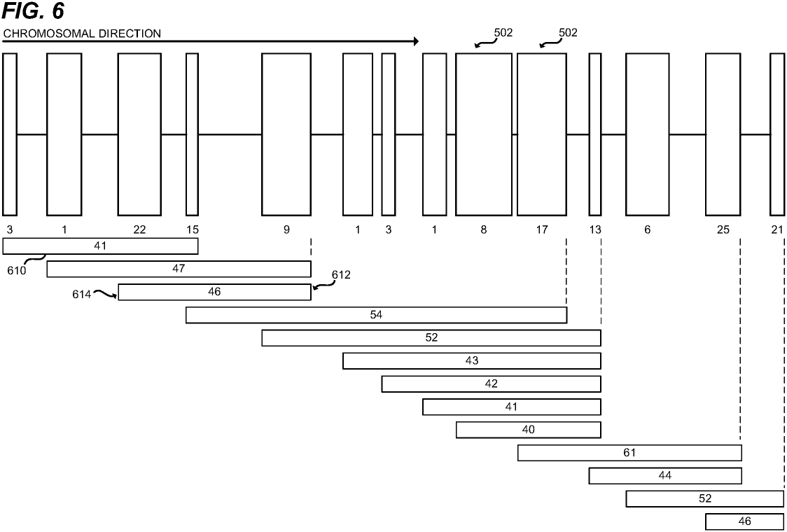

FIG. 6 depicts a sliding window 610 that slides across the portion 520 of the chromosome in an illustrative embodiment. In this embodiment, the sliding window is sized to include at least forty individuals carrying qualifying variants at any point in time. The sliding window 610 initially bridges four of the exons 502, encompassing qualifying variants carried by a total of forty-one individuals, and advances to a next position where it encompasses forty-seven individuals. In a next position, the sliding window 610 encompasses forty-six individuals, and so on as the sliding window advances from left to right in a chromosomal direction along the portion 520. The number of exons 502 encompassed by the sliding window 610 therefore varies as the sliding window 610 advances. In this embodiment, each position of the sliding window 610 is defined as a range of genomic coordinates, and the sliding window 610 is moved across thirteen positions.

In one embodiment, the controller 114 moves the sliding window 610 by adjusting a rear border 614 of the sliding window from a first exon 502 to an adjacent exon 502 along the portion 520 of the chromosome, and adjusting a front border 612 of the sliding window a variable number of exons 502 along the portion 520 of the chromosome. The number is variable because it depends upon how many persons in the population carry a qualifying variant for each exon 502. In a further embodiment, the sliding window 610 is moved systematically (e.g., base by base) across the portion 520, and is stopped for analysis upon reaching a sampling threshold.

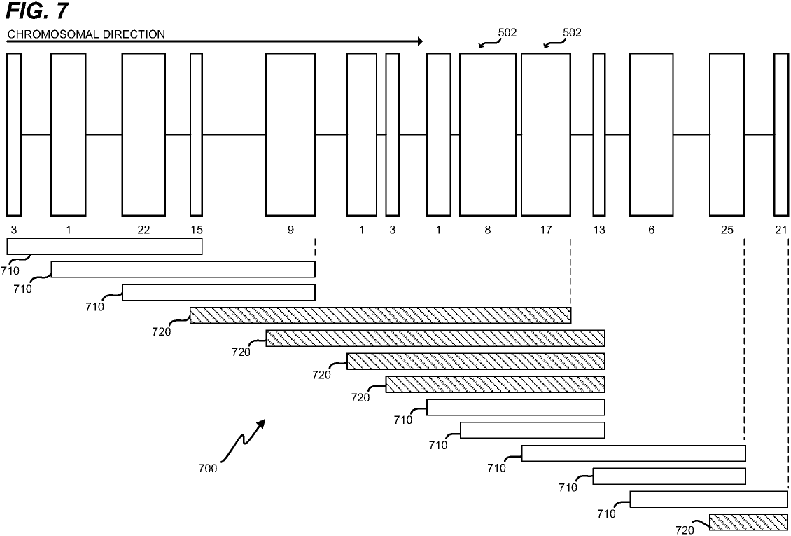

FIG. 7 depicts positions 700 of the sliding window 610 that are positively associated with a trait being considered in an illustrative embodiment. In this embodiment, the sliding window 610 has been analyzed at positions 710 that are not correlated with the trait being considered, and at other positions 720 that are correlated with the trait, as determined via the techniques discussed above with regard to FIG. 2.

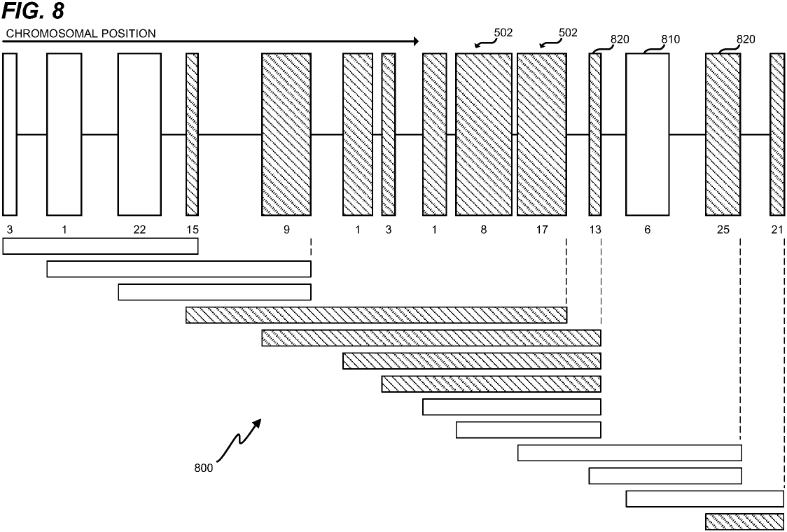

FIG. 8 depicts a mapping 800 of positions of the sliding window to qualifying variants in an illustrative embodiment. In FIG. 8, all exons covered by a sliding window 610 at a position 720 that is correlated with the trait are highlighted. This results in specific exons 820 being correlated with the trait, and other specific exons 810 not being correlated with the trait.

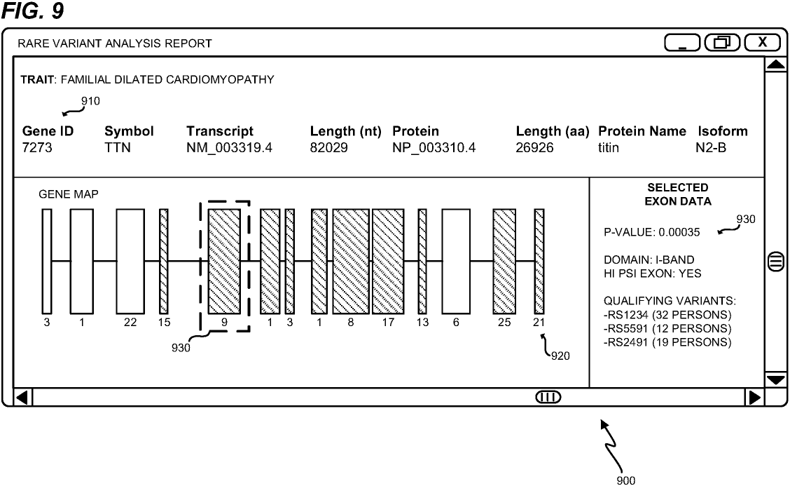

FIG. 9 depicts a Graphical User Interface (GUI) 900 that provides a report for review by a researcher in an illustrative embodiment. In this embodiment, the GUI 900 is subdivided into regions. Region 910 reports gene identifiers, or other identifying information for the portion of the chromosome that was investigated by a sliding window, as well as the trait being considered. Region 920 visually depicts exons (or variants, base pairs, etc.) that have been determined to be correlated with the trait being considered, and provides for selection of one or more exons (or variants, base pairs, etc.). Region 930 depicts the P-value calculated for the selected exon (or variant, base pair, etc.), a domain (e.g., I-band) for the selected exon (or variant, base pair, etc.), and whether or not the selected exon (or variant, base pair, etc.) is a hiPSI exon. Region 930 also provides data describing qualifying variants found within the selected exon (or variant, base pair, etc.).

### EXAMPLES

In the following examples, additional processes, systems, and methods are described in the context of a variant analysis system 100. The variant analysis system 100 investigates for correlations between the TTN gene and cardiac conditions.

In this example, controller 114 acquires sequence data 132 in the form of United Kingdom Biobank (UKB) plink-formatted population level exome Original Quality Functional Equivalence (OQFE) exome files from genomic database 130, for a population of two hundred thousand individuals. Controller 114 also acquires sequence data 132 in the form of imputed genotypes from GWAS genotyping. In this example, the sequence data 132 includes trait data 134 reporting ages and sexes of the population. Controller 114 refrains from performing filtering of the sequence data based on ancestry.

Next, controller 114 acquires additional trait data 134 in the form of medical records from genomic database 130 for the population. Controller 114 translates the medical records to phecodes based on logic stored in memory 112. Phecodes are translations of medical record data to phenotype data, and the techniques of translating the medical records to phecodes may comprise those established in existing published literature (e.g., using Phecode maps in the published literature, such as Wu P, Gifford A, Meng X, et al. Mapping ICD-10 and ICD-10-CM Codes to Phecodes: Workflow Developmentand Initial Evaluation. JMIR Med Inform 2019;7:e14325). During the translation process, phecodes may be collected from available diagnosis tables (e.g., from problem lists, medical histories, admissions data, surgical case data, account data, claims and invoices).

ICD codes and associated dates are collected as well by controller 114, as a part of this process. In this example, the ICD codes include ICD-9 (Phecode Map 1.2), ICD-10 (determined by Phecode Map 1.2b to ICD-10 beta), and ICD-10-CM (determined by Phecode Map 1.2b to ICD-10-CM beta) from the Phewas catalog to map ICDs to phecodes. When multiple ICDs from the same phecode are present for an individual, the earliest ICD date (e.g., to year) is used by controller 114 to represent that phecode. To normalize ICD dates, controller 114 transforms these dates to age at diagnosis, using the difference between the ICD date and birth year of each individual.

In the next step, controller 114 merges significant phecodes into a combined cardio phenotype. For genetic association analyses, phenotype matrices are constructed by coding 1 or 0 for presence or absence of any of seven phecodes that have shown statistically significant associations with the TTN gene in previously published rare-variant genome-wide analysis: primary/intrinsic cardiomyopathies, heart failure Not Otherwise Specified (NOS), Atrial Fibrillation and flutter (AFib), Congestive Heart Failure (CHF) NOS, nonrheumatic mitral valve disorders, mitral valve disease, and tachycardia NOS. This forms a “combined cardio” phenotype used for the main genetic association analyses in the present example. The aggregation/collection of the seven phecodes may be used as the trait that will be considered by controller 114 for correlation with qualifying variants. Any individual expressing at least one of the phecodes will be considered to be expressing a narrowly defined version of the trait.

In this example, controller 114 also defines extended cardio phenotypes in order to assess the severity and broader clinical impact of truncating variants in the TTN gene (TTNtvs). The extended cardio phenotypes include a TTN-related heart condition phenotype which gathers all heart-related phecodes in the Phecode Map surrounding the seven cardio phecodes discussed above. Candidate phecodes include those that have odds ratios consistent with an association with TTN (e.g., an odds ratio of greater than one). Controller 114 loads data that subdivides the candidate phecodes into lower severity (LS) and higher severity (HS) categories. LS diagnoses represent early to mid-stage outcomes that alone may not require immediate intervention as well as those that could not be assessed for severity without additional details. HS diagnoses represent diagnoses or procedures that signify serious risk of end-stage outcomes without timely intervention or are themselves end-stage outcomes or interventions to prevent end-stage outcomes. The combination of the seven phecodes and the candidate phecodes forms a broadly defined trait that can be considered for correlation with qualifying variants within a sliding window generated by controller 114. Any individual expressing at least one of the seven phecodes and/or any of the candidate phecodes is considered to be expressing the broadly defined trait. However, in some versions of this example, there is no need to re-run statistical analysis of the broadly defined trait, and the broadly defined trait is used for the purpose of better understanding associations detected for the narrowly defined trait.

Controller 114 also filters out the sequence data 132 and trait data 134 describing individuals with less than one year of diagnosis history, assessed by comparing the earliest and latest dates of any ICD code on record. This ensures that only individuals with a full history of diagnoses will be considered as a part of the analysis.

Next, controller 114 engages in annotation and Percent Spliced In (PSI) analysis. Specifically, controller 114 performs variant annotation with code for an Ensembl Variant Effect Predictor (VEP). In this example, coding regions are defined according to Gencode comprehensive gene annotation, version GENCODE 33, prepared by the GENCODE group which was established by the National Human Genome Research Institute (NHGRI), however other techniques may be utilized as desired. The Ensembl canonical transcript, published by the Ensembl Project, is used to determine variant consequence. Controller 114 restricts variants to Coding DNA Sequence (CDS) regions plus essential splice sites. Genotype processing is performed by controller 114 operating the Hail 0.2.54-8526838bf99f open-source library for scalable data exploration and analysis, relating to genotype processing and prepared by the Hail Team funded by the Broad Institute.

For the purposes of collapsing analysis, controller 114 codes the sequence data for an individual with a “1” if the individual carries a TTNtv and a “0” otherwise. In this example, TTNtv is defined as any LoF variant. Controller 114 further identifies qualifying variants for consideration. Variants are only included by controller 114 as qualifying variants if their MAF is below 0.1% in all gnomAD populations as well as locally within each population analyzed.

PSI data is obtained by controller from a website, such as cardiodb.org. The controller 114 obtains both left ventricle Dilated Cardiomyopathy (DCM) and Genotype-Tissue Expression (GTEx) values for heart tissue for each exon, and chooses the maximum value per exon as the value to use for inclusion in the PSI model. Exons with PSI > 90% are considered hiPSI by the controller 114 in this example. Furthermore, in this example, controller 114 also annotates LoF variants as either Low Confidence (LC) or high confidence (HC) according to LOFTEE30 standards.

Next, controller 114 generates a sliding window. The sliding window is moved to create a continuous pathogenicity model across the entire TTN locus. The basic concept of a sliding window analysis is to group variants located near each other into one unit and analyze them together to improve power, much like a gene-based collapsing analysis but at a smaller scale. Conceptually, this process of collapsing analysis may be considered to be performing three types of collapsing. The first type is performed at the gene level, taking all LoF variants (e.g., all exons) in TTN, the second type is performed by taking all LoF variants in just hiPSI exons (a subset of exons highly expressed in the heart), and the third type is performed by moving the sliding window to pick a subset of the hiPSI exons that have evidence of an association with the cardiac traits discussed above.

In this instance, rather than the size of the sliding window by the number of variants or bases remaining constant, the sliding window is moved to maintain roughly the same number of people with a rare TTNtv, which in this case constitutes a qualifying variant. Thus, the statistical power within the sliding window at each position along the TTN locus remains stable. When a single qualifying variant is well powered on its own, controller 114 removes the single qualifying variant out for its own separate analysis, and the sliding window slides past it, continuing to group surrounding variants as appropriate.

In this example, any TTN analysis window with at least forty carriers of a qualifying variant exhibits 80% power to identify an odds ratio of 2.9 with a p-value of 0.05, and 80% power to identify an odds ratio of 4 with a p-value of 0.001, similar to what is seen for rare TTNtvs in exons with PSI>90 (odds ratio=3.5). With forty qualifying variant carriers within each position of the sliding window, the power for discovery is the same for each position of the sliding window as it slides across the gene. Bases that fall between the boundaries of an associated position for the sliding window are assigned the same value in the testing set as that window has in the training set. Thus, new mutations that do not occur in the original dataset can still be assigned a value based on their location in the testing set. This also means that exons with no variation in the training set are assigned a value based on the values of their surrounding exons, which defines whether or not they are within the boundaries of an associated window. In this example, each position of the sliding window includes on average 21.4 variants (median 22, range 1-40) and 9.4 exons (median 9, range 1-25). Variants are on average 135.9 coding bases apart (median 84, range 0-1125).

Next, controller 114 engages in genetic analysis. In this embodiment, the controller 114 uses the Regenie code library for genetic analyses. Briefly, this method builds a whole genome regression model using common variants to account for the effects of relatedness and population stratification, and it accounts for situations where there is an extreme case-control imbalance, which can lead to test statistic inflation with other analysis methods. The covariates included by controller 114 as a part of this process are age, sex, age*sex, age*age, sex*age*age, and bioinformatics pipeline version. In this example, a representative set of 184,445 coding and noncoding LD-pruned, high-quality common variants are identified for building the whole genome regression model.

Controller 114 analyzes all ancestries together in this example, because when collapsing rare (MAF<0.1%) causal variants across a gene and analyzing with a linear mixed model or whole genome regression, signals tend to be consistent whether restricting to one ancestry or analyzing across all ancestries. This method works in this setting because analyses of collapsed rare variants are less influenced by demographic background than are analyses of the common variants used in a typical GWAS, in large part because causal variants are being grouped together as opposed to tagging variants.

Controller 114 performs separate analyses for the narrowly defined trait and the broadly defined trait for the entire TTN gene, as well as for the combined cardio phenotype in just hiPSI exons and the regions highlighted by the power window method. Controller 114 further performs meta-analysis across any cohorts in the sequence data (e.g., separate subpopulations sourced from different entities, or describing different sets of persons) using the weighted Z-score p-value in the METAL code library on the summary statistics from each separate analysis. The METAL code library facilitates meta-analysis of associational analyses, and is discussed in Willer, Cristen J et al. “METAL: fast and efficient meta-analysis of genomewide association scans.” Bioinformatics (Oxford, England) vol. 26,17 (2010): 2190-1. doi:10.1093/bioinformatics/btq340.

Next, controller 114 engages in statistical analyses. Outside of the main genetic analyses in the regenie code library, regression analyses are used for statistical analysis using the statsmodel code package in python. For binary variables, logistic regression is used; for count data, negative binomial regression is used; for quantitative variables, linear regression is used after rank-based inverse normal transformation of the variable. Time to event analysis is performed using the lifelines KaplanMeier package in python.

Having identified qualifying variants correlated with the narrowly defined trait, broadly defined trait, etc., controller 114 proceeds to generate a report for display to a researcher, indicating the outcome of the analyses. In this manner, controller 114 may actively highlight and indicate specific qualifying variants that may be desired for further investigation by the researcher.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

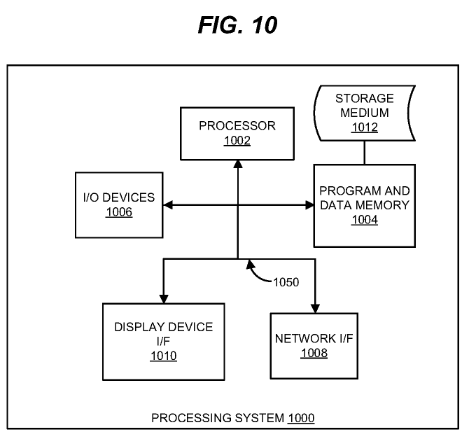

In one particular embodiment, instructions stored on a computer readable medium direct a computing system of any of the devices and/or servers discussed herein, such as analysis server 110, to perform the various operations disclosed herein. FIG. 10 depicts an illustrative computing system 1000 operable to execute a computer readable medium embodying programmed instructions. Computing system 1000 is operable to perform the above operations by executing programmed instructions tangibly embodied on computer readable storage medium 1012. In this regard, embodiments may utilize instructions (e.g., code) accessible via computer-readable medium 1012 for use by computing system 1000 or any other instruction execution system. For the purposes of this description, computer readable medium 1012 comprises any physical media that is capable of storing a program for use by computing system 1000. For example, computer-readable medium 1012 may be an electronic, magnetic, optical, electromagnetic, infrared, semiconductor device, or other non-transitory medium. Examples of computer-readable medium 1012 include a solid-state memory, a magnetic tape, a removable computer diskette, a random access memory (RAM), a read-only memory (ROM), a rigid magnetic disk, and an optical disk. Current examples of optical disks include Compact Disk -Read Only Memory (CD-ROM), Compact Disk - Read/Write (CD-R/W), Digital Video Disc (DVD), and Blu-Ray Disc.

Computing system 1000, which stores and/or executes the instructions, includes at least one processor 1002 coupled to program and data memory 1004 through a system bus 1050. Program and data memory 1004 include local memory employed during actual execution of the program code, bulk storage, and/or cache memories that provide temporary storage of at least some program code and/or data in order to reduce the number of times the code and/or data are retrieved from bulk storage (e.g., a spinning disk hard drive) during execution.

Input/output or I/O devices 1006 (including but not limited to keyboards, displays, touchscreens, microphones, pointing devices, etc.) may be coupled either directly or through intervening I/O controllers. Network adapter interfaces 1008 may also be integrated with the system to enable computing system 1000 to become coupled to other computing systems or storage devices through intervening private or public networks. Network adapter interfaces 1008 may be implemented as modems, cable modems, Small Computer System Interface (SCSI) devices, Fibre Channel devices, Ethernet cards, wireless adapters, etc. Display device interface 1010 may be integrated with the system to interface to one or more display devices, such as screens for presentation of data generated by processor 1002.

What is claimed is:

1. A system comprising:
a memory configured to store sequence data for a portion of a chromosome that indicates, for each individual in a population, variants found in the individual within the portion of the chromosome, and trait data indicating, for each of the individuals, an extent that the individual expresses a predefined trait controlled by the portion of the chromosome; and
a controller configured to identify qualifying variants within the sequence data, the qualifying variants comprising variants that meet criteria for analysis, and for each qualifying variant, to determine a genomic coordinate of the qualifying variant at the portion of the chromosome, as well as a number of the individuals in the population carrying the qualifying variant,
the controller is further configured to generate a sliding window comprising a selection of a sequential set of variants within the portion of the chromosome, wherein a number of the individuals in the population carrying a qualifying variant at the sliding window is within a predetermined range, and to iteratively:
perform a statistical analysis that indicates whether qualifying variants at genomic coordinates within a region occupied by the sliding window are correlated with the trait, based on a comparison of trait data for individuals carrying the qualifying variant to trait data for individuals in the population, and
move the sliding window across at least one variant along a chromosomal direction, while dynamically adjusting a number of variants encompassed by the sliding window to maintain a number of the individuals in the population carrying a qualifying variant at the sliding window within the predetermined range,
the controller is further configured to selectively categorize qualifying variants as correlated with the trait, based on the statistical analysis, and to report qualifying variants correlated with the trait to a user via a display.

2. The system of claim 1 wherein:
the controller is further configured to move the sliding window by adjusting a rear border of the sliding window from a first variant to an adjacent variant along the portion of the chromosome, and adjusting a front border of the sliding window a variable number of variants along the portion of the chromosome.

3. The system of claim 1 wherein:
the controller is further configured to compare results of the statistical analysis for the sliding window at different regions, and to identify at least one region within the portion of the chromosome that is more highly correlated with the trait than other regions within the portion of the chromosome.

4. The system of claim 1 wherein:
the controller is further configured to alter the predetermined range, generate a new sliding window, and iteratively perform statistical analysis and move the new sliding window according to the altered predetermined range, and
the controller is further configured to report qualifying variants correlated with the trait for both the predetermined range and the altered predetermined range.

5. The system of claim 1 wherein:
the statistical analysis comprises determining a first ratio of individuals expressing the trait among individuals carrying the qualifying variant, determining a second ratio of individuals expressing the trait among the population, and determining an odds ratio based on: a comparison of the first ratio to the second ratio, a number of individuals carrying the qualifying variant, a number of individuals in the population, and consideration of covariates that affect the predefined trait.

6. The system of claim 1 wherein:
the range includes a number which ensures that an odds ratio is calculated to a predetermined margin of error at each position of the sliding window.

7. The system of claim 1 wherein:
the chromosomal direction is at least one of a 5′ to 3′ direction, or a 3′ to 5′ direction along variants in the portion of the chromosome.

8. The system of claim 1 wherein:
variants that meet the criteria for analysis comprise variants that alter a structure of a protein generated by the portion of the chromosome.

9. A method comprising:
storing sequence data for a portion of a chromosome that indicates, for each individual in a population, variants found in the individual within the portion of the chromosome;
storing trait data indicating, for each of the individuals, an extent that the individual expresses a predefined trait controlled by the portion of the chromosome;
identifying qualifying variants within the sequence data, the qualifying variants comprising variants that meet criteria for analysis;
for each qualifying variant, determining a genomic coordinate of the qualifying variant at the portion of the chromosome, as well as a number of the individuals in the population carrying the qualifying variant,
generating a sliding window comprising a selection of a sequential set of variants within the portion of the chromosome, wherein a number of the individuals in the population carrying a qualifying variant at the sliding window is within a predetermined range;
iteratively:
performing a statistical analysis that indicates whether qualifying variants at genomic coordinates within a region occupied by the sliding window are correlated with the trait, based on a comparison of trait data for individuals carrying the qualifying variant to trait data for individuals in the population, and
moving the sliding window across at least one variant along a chromosomal direction, while dynamically adjusting a number of variants encompassed by the sliding window to maintain a number of the individuals in the population carrying a qualifying variant at the sliding window within the predetermined range;
selectively categorizing qualifying variants as correlated with the trait, based on the statistical analysis; and
reporting qualifying variants correlated with the trait to a user via a display.

10. The method of claim 9 further comprising:
moving the sliding window by adjusting a rear border of the sliding window from a first variant to an adjacent variant along the portion of the chromosome, and adjusting a front border of the sliding window a variable number of variants along the portion of the chromosome.

11. The method of claim 9 further comprising:
comparing results of the statistical analysis for the sliding window at different regions; and
identifying at least one region within the portion of the chromosome that is more highly correlated with the trait than other regions within the portion of the chromosome.

12. The method of claim 9 further comprising:
altering the predetermined range, generate a new sliding window;
iteratively performing statistical analysis and move the new sliding window according to the altered predetermined range; and
reporting qualifying variants correlated with the trait for both the predetermined range and the altered predetermined range.

13. The method of claim 9 wherein:
the statistical analysis comprises determining a first ratio of individuals expressing the trait among individuals carrying the qualifying variant, determining a second ratio of individuals expressing the trait among the population, and determining an odds ratio based on: a comparison of the first ratio to the second ratio, a number of individuals carrying the qualifying variant, a number of individuals in the population, and consideration of covariates that affect the predefined trait.

14. The method of claim 9 wherein:
the range includes a number which ensures that an odds ratio is calculated to a predetermined margin of error at each position of the sliding window.

15. The method of claim 9 wherein:
the chromosomal direction is at least one of a 5′ to 3′ direction, or a 3′ to 5′ direction along variants in the portion of the chromosome.

16. The method of claim 9 wherein:
variants that meet the criteria for analysis comprise variants that alter a structure of a protein generated by the portion of the chromosome.

17. A non-transitory computer readable medium embodying programmed instructions which, when executed by a processor, are operable for performing a method comprising:
storing sequence data for a portion of a chromosome that indicates, for each individual in a population, variants found in the individual within the portion of the chromosome;
storing trait data indicating, for each of the individuals, an extent that the individual expresses a predefined trait controlled by the portion of the chromosome;
identifying qualifying variants within the sequence data, the qualifying variants comprising variants that meet criteria for analysis;
for each qualifying variant, determining a genomic coordinate of the qualifying variant at the portion of the chromosome, as well as a number of the individuals in the population carrying the qualifying variant,
generating a sliding window comprising a selection of a sequential set of variants within the portion of the chromosome, wherein a number of the individuals in the population carrying a qualifying variant at the sliding window is within a predetermined range;
iteratively:
performing a statistical analysis that indicates whether qualifying variants at genomic coordinates within a region occupied by the sliding window are correlated with the trait, based on a comparison of trait data for individuals carrying the qualifying variant to trait data for individuals in the population, and
moving the sliding window across at least one variant along a chromosomal direction, while dynamically adjusting a number of variants encompassed by the sliding window to maintain a number of the individuals in the population carrying a qualifying variant at the sliding window within the predetermined range;
selectively categorizing qualifying variants as correlated with the trait, based on the statistical analysis; and
reporting qualifying variants correlated with the trait to a user via a display.

18. The computer readable medium embodying programmed instructions of claim 17, wherein the instructions are operable for performing a method further comprising:
moving the sliding window by adjusting a rear border of the sliding window from a first variant to an adjacent variant along the portion of the chromosome, and adjusting a front border of the sliding window a variable number of variants along the portion of the chromosome.

19. The computer readable medium embodying programmed instructions of claim 17, wherein the instructions are operable for performing a method further comprising:
comparing results of the statistical analysis for the sliding window at different regions; and
identifying at least one region within the portion of the chromosome that is more highly correlated with the trait than other regions within the portion of the chromosome.

20. The computer readable medium embodying programmed instructions of claim 17, wherein the instructions are operable for performing a method further comprising:
altering the predetermined range, generate a new sliding window;
iteratively performing statistical analysis and move the new sliding window according to the altered predetermined range; and
reporting qualifying variants correlated with the trait for both the predetermined range and the altered predetermined range.
